(19) United States
(12) Patent Application Publication
(10) Pub. No.: US 2011/0033818 A1
(43) Pub. Date: Feb. 10, 2011

(54) INTRAORAL TOOL CLEANING SYSTEM AND METHOD

(76) Inventor: Robert Fenwick Miller, Walnut Creek, CA (US)

Correspondence Address: Robert Fenwick Miller, 1181 Boulevard Way, #C, Walnut Creek, CA, 94549, US

(21) Appl. No.: 12/536,380

(22) Filed: Aug. 5, 2009

(51) Int. Cl. B08B 1/00; A61B 1/247

(52) U.S. Cl. 433/31; 15/104.92

(57) ABSTRACT

An intraoral tool cleaning system and method of use of the system, enabling a user to clean debris and fog from an intraoral mirror with a one-handed motion and limited cross-contamination between patients. In one embodiment, the system includes a disposable sponge adapted to remove particles from a mirror surface of the intraoral mirror by abrading the mirror surface, a housing that contains the sponge, and a base that secures the housing to a surface. In another embodiment, the system includes a disposable sponge, a disposable base that secures the sponge to a surface on an appendage of the user, and a fastener that adheres the sponge to the base.

### TECHNICAL FIELD

This invention relates generally to the dental field, and more specifically to an intraoral tool cleaning system and method.

### BACKGROUND

During dental procedures, a dentist typically uses an intraoral mirror to gain visual of areas of the mouth that would otherwise be difficult to see. However, the mirror often becomes fogged and/or covered by debris such as blood, dental particles, and filling particles, which significantly reduce visibility and make dental procedures more difficult. The problem of overcoming objectionable fogging and clearing debris from an intraoral mirror during a dental operation has given rise to several mirror-cleaning technologies, including intraoral mirror cleaning systems and self-cleaning intraoral mirrors.

Current intraoral mirror cleaning systems involve dipping the mirror surface of the intraoral mirror into a sterilizing solution or surfactant bath and/or wiping the mirror surface on a wick. However, these current systems have drawbacks. Dipping the mirror surface into a bath does not sufficiently remove particles from the mirror surface. Wiping the mirror surface on a wick often demands use of two hands, which interrupts procedural flow by requiring the dentist or dental assistant to set down hand-held tools to clean the mirror.

Current self-cleaning intraoral mirrors utilize relative mechanical motion between the mirror and a wiper blade. These systems may also incorporate a hollow handle and a perforated mirror through which an externally supplied fluid, such as water or air, may pass to allow continuous cleaning to the mirror surface. However, these systems do not reliably prevent cross-contamination between patients, and have been difficult to integrate into real world settings due to their complexity and cost.

Thus, there is a need in the dental field to create an improved intraoral tool cleaning system that accommodates the effective removal of fog and debris without disrupting procedural flow or increasing cross-contamination between patients or other indirect users.

### BRIEF DESCRIPTION OF THE FIGURES

FIGS. 1 and 2 are side and front views, respectively, of the first preferred embodiment of the intraoral tool cleaning system;

FIG. 3 is a partial side view of the housing of the first preferred embodiment of the intraoral tool cleaning system, with the side opening hidden for clarity;

FIG. 4 is a perspective view of the housing of the first preferred embodiment of the intraoral tool cleaning system;

FIGS. 5A and 5B are side views of the housing and the furrow of the base engaging in a tongue-and-groove joint variation and an inverted “T”-shape joint variation, respectively;

FIGS. 6A and 6B are front and side views, respectively, of the base of the first preferred embodiment of the intraoral tool cleaning system;

FIGS. 7A and 7B are front and closeup views of the second preferred embodiment of the intraoral tool cleaning system;

FIGS. 8 and 9 are views of a first variation and a second variation, respectively, of the second preferred embodiment of the intraoral tool cleaning system;

FIG. 10 is a flowchart depicting one variation of the method of the intraoral tool cleaning system; and

FIG. 11 is a flowchart depicting another variation of the method of the intraoral tool cleaning system.

### DESCRIPTION OF THE PREFERRED EMBODIMENTS

The following description of the preferred embodiment of the invention is not intended to limit the invention to this preferred embodiment, but rather to enable any person skilled in the art to make and use this invention.

The intraoral tool cleaning system of the preferred embodiment has been specifically designed to enable a user to remove particles such as water particles, blood and other fluid particles, and dental and filling debris from a mirror surface of an intraoral mirror using a one-handed motion repeatedly as needed during a dental operation. The system may additionally and/or alternatively be used to remove particles from any suitable surface from any suitable intraoral tool, such as a pick, water gun, air gun, or suction nozzle. The system preferably limits disruption of procedural flow during a dental operation and limits cross-contamination between patients or other indirect users.

### 1. First Preferred Embodiment of the Intraoral Tool Cleaning System

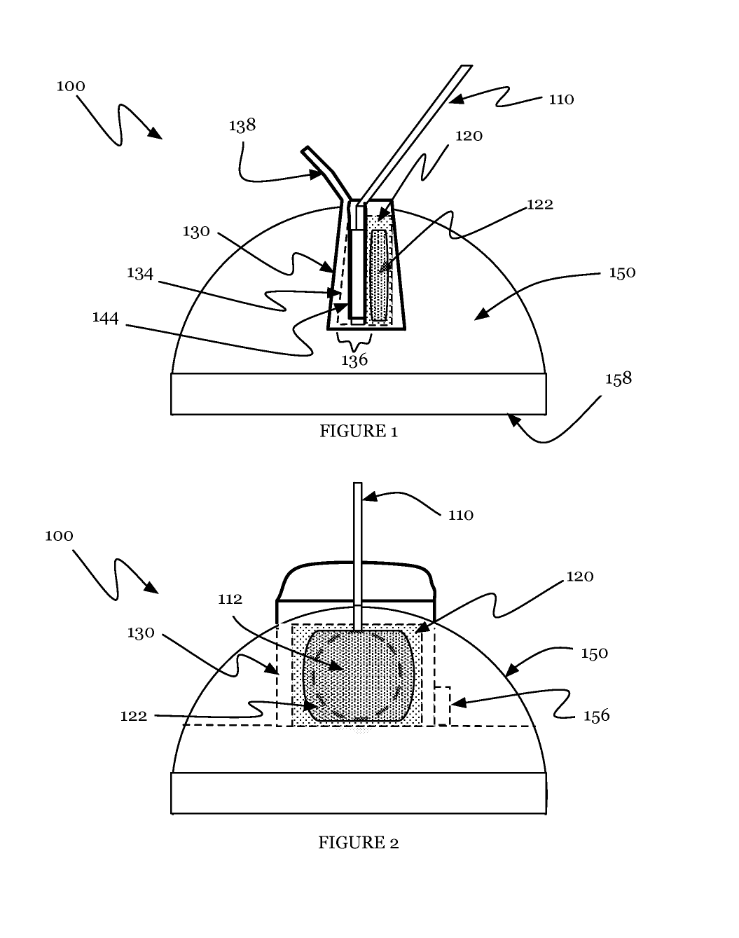

As shown in FIGS. 1 and 2, the intraoral tool cleaning system 100 of the first preferred embodiment includes a sponge 120 that removes particles from a mirror surface 112 of an intraoral mirror 110 by abrading the mirror surface 112, a housing 130 that contains the sponge 120, and a base 150 that secures the housing to a surface.

As shown in FIG. 1, the sponge 120 of the first preferred embodiment functions to remove particles from a mirror surface 112 by abrading the mirror surface. The sponge 120 may alternatively remove particles from any suitable surface of any suitable intraoral tool. The sponge 120 preferably removes particles when a user applies the mirror surface 112 to the sponge 120 in a one-handed motion such as rubbing, wiping, swabbing, or any suitable one-handed motion. The sponge 120 may alternatively remove particles from the surface 112 with any suitable user motion. The one-handed motion is preferably performed by the hand of the user that holds the intraoral mirror 110 during a dental operation executed by the user, such that the user can clean the mirror surface quickly and easily, with minimal interruption of the dental operation. The sponge 120 is preferably saturated with a surfactant fluid 122 that helps maintain a clear visual on the mirror surface when applied to the mirror surface. The surfactant fluid 122 is preferably a disinfectant, detergent, enzymatic cleaner or hydrophilic surfactant, but may alternatively be any suitable fluid that cleans and defogs the mirror surface. Alternatively, the sponge 120 of the first preferred embodiment may include no fluid, and/or any suitable means for altering any suitable surface in any desired manner, such as by modifying surface tension or chemical composition. The sponge 120 may include adhesive or another securement mechanism to secure the sponge 120 in the housing 130.

The sponge 120 preferably has a cleaning surface that is equal in area to or larger in area than the mirror surface of a typical intraoral tool, such that the sponge takes longer to become fully saturated with debris, as the mirror surface 112 is repeatedly cleaned with the sponge 120 throughout a dental operation. The sponge 120 may alternatively and/or additionally be rinsable, such that rinsing the sponge removes debris from the sponge as needed, thereby prolonging the useful life of the sponge. The sponge 120 is preferably generally flat to uniformly clean a mirror surface, but may alternatively be concave, convex, and/or have any suitable geometry to clean any suitable surface. The sponge 120 is preferably disposable, so that it can be discarded after it becomes fully saturated with debris and is unusable, or discarded after the dental operation on one patient is complete. Discarding the sponge, which is direct contact with bodily fluids, limits cross-contamination between patients. The sponge 120 is preferably made of an absorbent, abrasive material, such as foam, cellulose, luffa or sea sponge, or any suitable disposable material that can be saturated with a fluid and/or provide texture to abrade the mirror surface 112.

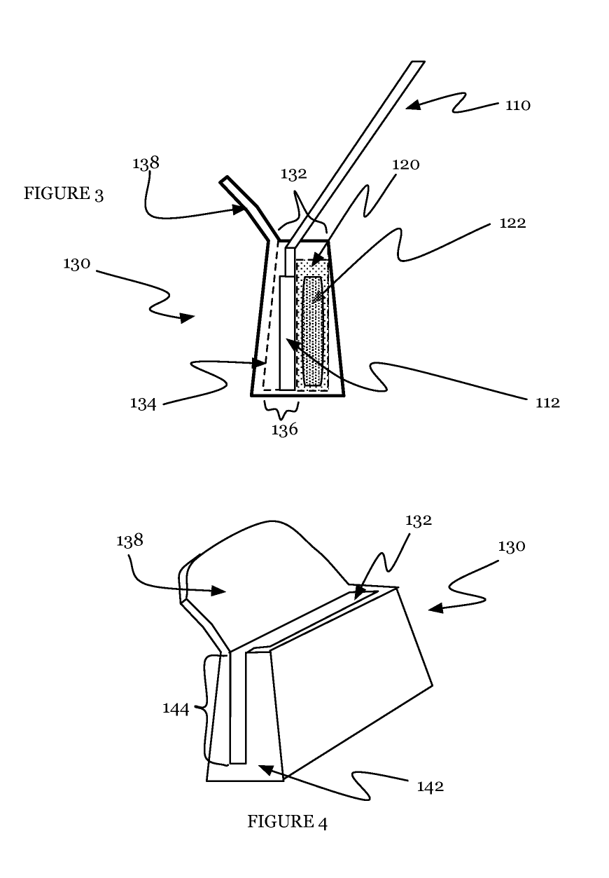

The housing 130 of the first preferred embodiment preferably functions to contain the sponge 120 and to accommodate an insertion of the mirror surface 112 into the housing. The sponge 120 may be secured within the housing 130 with adhesive or friction, or with any suitable securement mechanism. The sponge 120 may be permanently fixed within the housing, or may be removable from the housing. As shown in FIG. 3, the housing 130 preferably includes an upper opening 132 and a back wall 134 adjacent to the sponge 120, such that the back wall 134 and sponge 120 define a vertical slot 136 that extends from the upper opening 132 into the housing 130. The upper opening 132 preferably functions as an entrance through which the mirror surface 112 is inserted into the vertical slot 136, and/or as an exit through which the mirror surface is removed from the vertical slot 136. The vertical slot 136 preferably has a thickness at least as thick as the mirror surface 112 to receive the mirror surface and to provide enough space that permits abrading motion between the mirror surface and the sponge.

The housing 130 preferably further includes a backrest 138 that extends from the back wall 134 and guides receiving of the mirror surface 112 into the vertical slot 136. The backrest 138 is preferably generally flat and angled away from the upper opening 132, to provide a surface against which the intraoral mirror 110 may slide to enter the vertical slot 136. The backrest 138 may be at an angle of 45 degrees or less from the vertical, to guide the mirror surface 112 into the vertical slot 136, but may alternatively be angled away from the upper opening 132 with any suitable slope, angled toward the upper opening, curved, and/or any suitable shape. The housing 130 may alternatively include two or more backrests that function together similar to a funnel to guide the mirror surface 112 into the vertical slot 136. The width of the vertical slot 136 is preferably at least as wide as the housing 130, to provide guidance of the mirror surface into the vertical slot 136 along the entire width of the housing 130.

As shown in FIG. 4, the housing 130 preferably further includes a side wall 142 with at least one side opening 144, such that the vertical slot 136 extends through the side opening 144. The side opening 144 preferably functions as an exit through which the mirror surface 112 is removed from the vertical slot 136, but may alternatively and/or additionally function as an entrance through which the mirror surface is inserted into the vertical slot 136. The width of the side opening 144 is preferably at least the thickness of a typical intraoral mirror no. In one variation, the housing 130 includes one side opening 144 positioned on a suitable side of the housing. For example, a user who typically holds the intraoral mirror 110 in their right hand may desire the side opening 144 to be positioned on the right-hand side of the housing 130, to allow for easier removal of the intraoral mirror no. In another variation, the housing 130 includes two side openings on opposite sides of the housing, to allow a more universal use of the system that is suitable for users who hold the intraoral mirror in their left hand and those who hold the intraoral mirror in their right hand. The housing may, however, include any suitable number, including zero, of side openings.

In a variation of the housing 130, the housing is similar to that of the preferred embodiment except that the vertical slot 136 is oriented horizontally or at another nonvertical angle.

The housing 130 is preferably made of a rigid or semi-rigid, leak-resistant, disposable material, such as plastic or waterproof paper, to allow the housing to be discarded. Similar to the sponge 120, the housing 130 is in direct contact with bodily fluids of the patient, so discarding the housing after use with one patient limits cross-contamination between patients. The housing 130 and sponge 120 are preferably discarded together after one use. In a variation, the housing 130 is made of a rigid or semi-rigid, leak-resistant, reusable material, such as stainless steel, to allow the housing to be sterilized after each use and reused for multiple patients. In this variation, the sponge 120 is preferably removed from the housing 130 and discarded before the housing is sterilized. Alternatively, the housing may also be made of any suitable material.

The base 150 of the first preferred embodiment functions to secure the housing 130 to another surface, such as a table. The base 150 preferably includes a housing fitting that retains the housing 130 and a slip-resistant surface 158 that resists movement of the base 150 relative to the surface. The surface is preferably a generally flat surface, such as a tabletop, tray, or chair armrest, but may alternatively be any suitable surface.

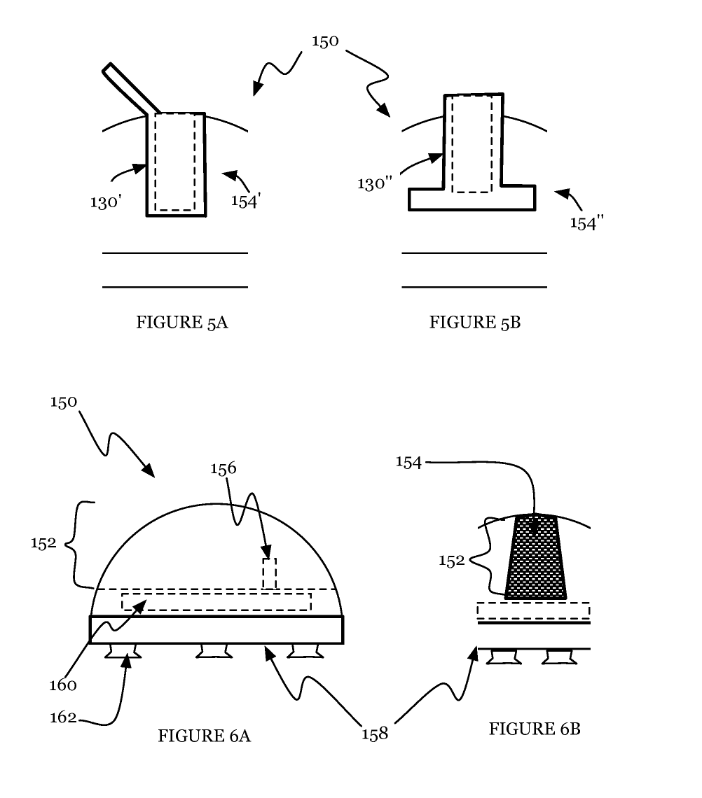

The housing fitting 152 of the base 150 preferably includes a furrow 154 that extends across the base and receives the housing 130 by selectively engaging with the housing in a sliding manner. As shown in FIG. 1, the furrow 154 preferably engages with the housing 130 in a sliding dovetail joint, in which the furrow 154 has a trapezoidal cross-section that enables the furrow to function as a dovetail slot and the housing has a trapezoidal cross-section that enables the housing to function as a dovetail pin. The furrow 154 may alternatively engage with the housing 130 with a tongue-and-groove joint (FIG. 5A), or any suitable sliding joint or friction fitting that limits separation of the housing from the furrow, in which the furrow 154 and the housing have complementary cross-sections. For example, the furrow 154 and the housing 130 may have cross-sections such as an inverted T-shape (FIG. 5B), an L-shape, a teardrop shape, or any suitable shape. The length of the furrow 154 is preferably equal to or longer than the width of the housing, such that the housing is fully engaged with the furrow for structural stability of the housing, but the length of the furrow may be any suitable length. Alternatively, the housing fitting 152 may include any suitable structure and/or mechanism that retains the housing, such as magnets, hook and loop fastener, epoxy, and/or any suitable fastener or adhesive.

The housing fitting preferably includes a retention stop 156 that limits the positional range in which the housing 130 may engage with the furrow 154. The retention stop 156 is preferably a ridge at the bottom of the furrow that blocks further sliding engagement between the housing 130 and the furrow 154, but may alternatively include a ridge on the side of the furrow, a wall, a tightening friction fit between the housing and the furrow, or any suitable structure and/or mechanism that limits the positional range of the housing 130.

As shown in FIGS. 6A and 6B, the slip-resistant surface 158 of the base 150 preferably includes a mechanism to resist movement of the base relative to the surface. The mechanism may include one or more silicone feet, a suction cup 162, a clamp, adhesive, straps, hook and loop fastener, and/or a magnet. The base 150 may alternatively and/or additionally be made of and/or include a weight of a high-density material 160, such as stainless steel or plastic coated lead, that weights the base 150 on the surface, and/or include geometry that hooks or wraps around a surface such as a patient chair armrest.

As shown in FIGS. 1 and 6A, the base 150 is preferably wider at its bottom than at its top, to increase stability of the base on the surface. The base is preferably overall a dome shape, but may alternatively be a truncated pyramid or any suitable shape.

### 2. Second Preferred Embodiment of the Intraoral Tool Cleaning System

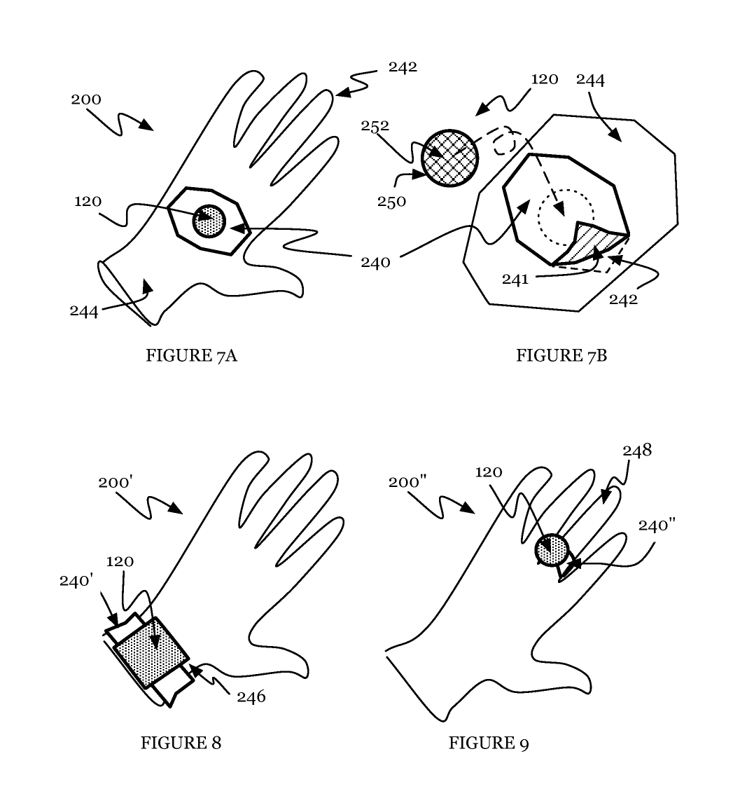

As shown in FIGS. 7A and 7B, the intraoral tool cleaning system 200 of the second preferred embodiment preferably includes a disposable sponge 120 adapted to remove particles from a mirror surface 112 of the intraoral mirror 110 by abrading the mirror surface; a base 240 that secures the sponge 120 to a surface; and a fastener 250 that attaches the sponge 120 to the base 240.

The disposable sponge 120 of the second embodiment is preferably identical to the disposable sponge 120 of the first embodiment.

The base 240 of the second embodiment preferably functions to secure the sponge 120 to a surface 242. The surface 242 is preferably located on an appendage of the user, such as on a hand, a finger, forearm, wrist, knee, or thigh of a user. Attachment of the base 240 to an appendage of the user increases accessibility of the system to the user. The base 240 is preferably disposable and intended for one use, but may alternatively be sterilizable and reusable for multiple uses. As shown in FIG. 7B, the base 240 preferably includes an adhesive patch 241 that adheres to a surface 242 on the outside of a medical glove 244, such as a disposable medical glove made from latex, vinyl or nitrile rubber. The adhesive patch 241 preferably covers a wide area, preferably on the back of the hand, to increase stability on the surface and to provide an accessible, sufficient area for the sponge 120 to be secured. However, the adhesive patch 241 may alternatively cover any suitable area on any suitable kind of glove. The adhesive patch 241 may be manufactured similar to conventional adhesive bandages, and may be stored in sealed packaging similar to conventional adhesive bandages. The base 240 may alternatively be integrated into the medical glove, such that the sponge is secured directly to the glove. In a first variation, as shown in FIG. 8, the base is a wrist strap 240′ worn on a surface on the wrist 246 of the user. The wrist strap 240′ may include elastic or a series of snaps or hook and eyes for an adjustable fit on the wrist, and/or may be manufactured as one of a series of available sizes. The wrist strap 240′ may be made of a disposable material for one-time use, or may be made of a reusable material suitable for multiple uses. Possible wrist strap materials include paper, synthetic fabric, plastic, elastic, and metal, but may alternatively and/or additionally include any suitable material. In a second variation, the base is a ring 240″ that is worn on a surface on a finger 248 of the user. The ring 240″ is preferably similar to the wrist strap 240′ of the first variation of the base, except that the ring is adapted to have an adjustable fit for the finger. Alternatively, the base 240 may secure the sponge 120 to any suitable stable surface through any suitable means, such as suction or clamping, or another kind of fastener.

As shown in FIG. 7B, the fastener 250 of the second preferred embodiment preferably functions to adhere the sponge 120 to the base 240. The fastener 250 preferably includes an adhesive material 252 that is applied to the sponge 120. The adhesive material 252 preferably includes epoxy, but may alternatively and/or additionally include a magnet, hook and loop fastener, a wax-based adhesive, or any suitable material that adheres the sponge to the base. The fastener 250 preferably permanently adheres the sponge 120 to the base 240, such that the sponge and base may be discarded together after one use. The fastener 250 may alternatively temporarily or selectively adhere the sponge 120 to the base 240, such that the sponge may be selectively separated from the base and discarded while the base is sterilized and reused. Alternatively, the fastener may connect the sponge 120 to the base 240 in any suitable permanent or removable manner and be made of any suitable material that enables the intended function.

In a variation of the second preferred embodiment, the system 200 may further include a housing similar to the housing 130 of the first embodiment, except that the fastener of the second preferred embodiment functions to secure the housing to the base. The housing of the second embodiment may include an open side instead of a vertical slot, to allow more accessibility to the sponge. As an example, the housing of the second preferred embodiment may be an open-top box that contains the sponge like a cradle.

### 3. Method of Providing and Using the Intraoral Tool Cleaning System

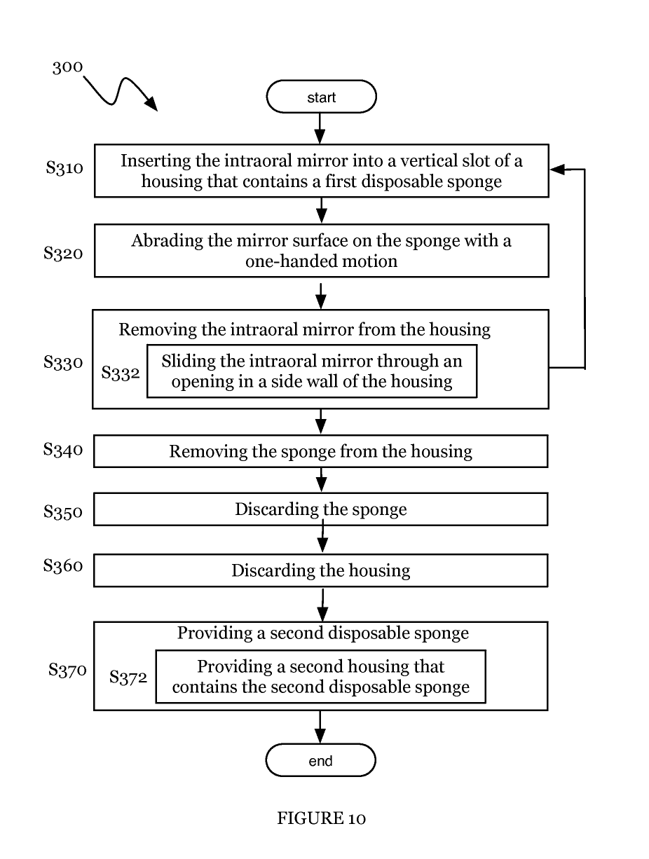

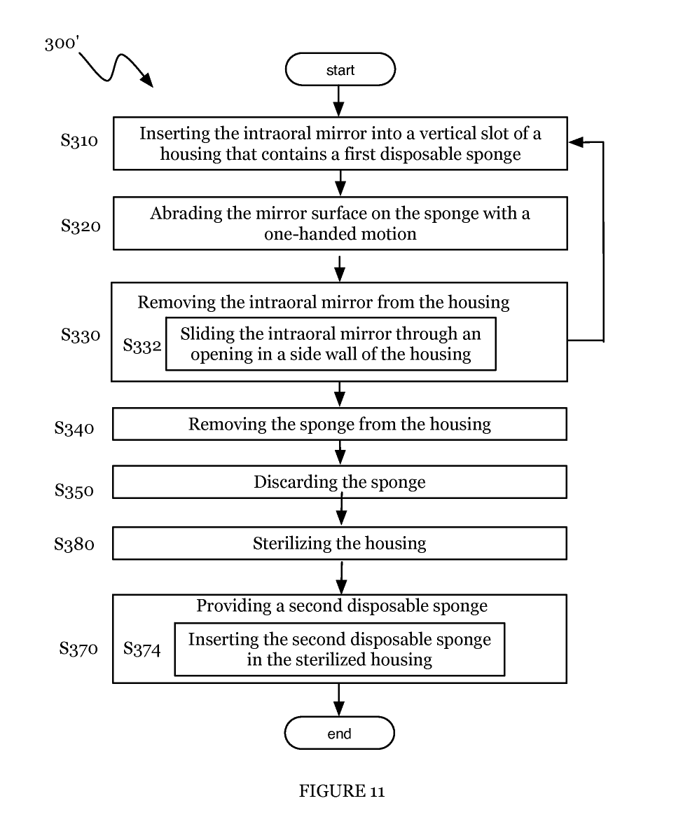

As shown in FIGS. 10 and 11, the method 300 of the preferred embodiment for cleaning debris and fog from a mirror surface of an intraoral mirror with a one-handed motion includes: inserting the intraoral mirror into a vertical slot of a housing that contains a first disposable sponge S310; abrading the mirror surface on the sponge with a one-handed motion such that the sponge removes particles from the mirror surface S320; removing the intraoral mirror from the housing S330; removing the sponge from the housing S340; discarding the sponge S350; and providing a second disposable sponge S370. The step of inserting the intraoral mirror into a vertical slot of a housing S310, abrading the mirror surface on the sponge S320, and removing the intraoral mirror from the housing S330 are preferably repeated periodically or as needed to clean the mirror surface during a dental operation. The method 300 is preferably performed to clean a mirror surface of an intraoral mirror, but may alternatively be performed to clean any suitable surface from any suitable intraoral tool, such as a pick, water gun, air gun, or suction nozzle. The method preferably limits disruption of procedural flow during a dental operation and limits cross-contamination between patients or other indirect users.

The step of inserting the intraoral mirror into a slot of a housing that contains a first disposable sponge S310 functions to bring the mirror surface into contact with the first disposable sponge. The slot is preferably defined by the sponge and a back wall adjacent to the sponge. The sponge is preferably saturated with a surfactant fluid and is adapted to remove particles from the mirror surface by abrading the mirror surface on the sponge.

The step of abrading the mirror surface on the sponge S320 functions to remove particles from the mirror surface. The step S320 is preferably performed with a rubbing, wiping, swabbing, or any suitable one-handed motion engendering relative motion between the mirror surface and the sponge. The one-handed motion is preferably performed by the hand of the user that holds the intraoral mirror during a dental operation executed by the user, such that the user can clean the mirror surface quickly and easily, with minimal interruption of the dental operation.

The step of removing the intraoral mirror from the housing S330 preferably includes sliding the intraoral mirror through an opening in a side wall of the housing S332. The opening in the side wall of the housing preferably functions as an exit through which the mirror surface is swiftly removed from the vertical slot, but may alternatively and/or additionally function as an entrance through which the mirror surface is inserted into the vertical slot.

The step of removing the sponge from the housing S340 is preferably performed after the sponge has become fully saturated with debris, and/or the dental operation on one patient is complete.

In one variation of the method, the method 300 further includes discarding the housing S360. The step of discarding the housing S350 may be performed after discarding the sponge S350, or may be performed simultaneously with discarding the sponge S350. In this variation, the step of providing a second disposable sponge S370 further includes providing a second housing that contains the second disposable sponge S372. In this variation, the sponge and the housing are preferably made of disposable materials intended for a one time use. Discarding the sponge and the housing, which directly contact bodily fluids of the patient, limits cross-contamination between patients.

In another variation of the method, the method 300′ further includes sterilizing the housing S38, preferably performed after discarding the sponge S350. In this variation, the step of providing a second disposable sponge S370′ includes inserting the second disposable sponge in the sterilized housing S374. Inserting a new, unused sponge into a clean housing limits cross-contamination between patients.

As a person skilled in the art will recognize from the previous detailed description and from the figures and claims, modifications and changes can be made to the preferred embodiment of the invention without departing from the scope of this invention defined in the following claims.

I claim:

1. An intraoral tool cleaning system that enables a user to clean debris and fog from an intraoral mirror with a one-handed motion, comprising:
a disposable sponge that removes particles from a mirror surface of the intraoral mirror by abrading the mirror surface, wherein the sponge is made of an absorbent foam material and is saturated with a surfactant fluid;
a rigid housing that contains the sponge, wherein the housing includes an upper opening and a back wall adjacent to the sponge; wherein the back wall and the sponge define a vertical slot that extends from the upper opening into the housing and is adapted to receive the mirror surface; wherein the vertical slot permits abrading motion between the mirror surface and the sponge; wherein the housing further includes a generally flat backrest extending from the back wall that guides receiving of the mirror surface into the vertical slot; wherein the backrest is angled away from the upper opening; and
a base that secures the housing to a surface, wherein the base includes a housing fitting that retains the housing; wherein the housing fitting includes a furrow that extends across the base and receives the housing by selectively engaging with the housing in a sliding manner; wherein the length of the furrow is at least equal to the width of the housing; wherein the base further includes a slip-resistant surface that resists movement of the base relative to the surface.

2. The intraoral tool cleaning system of claim 1, wherein the vertical slot of the housing extends through an opening in a side wall of the housing.

3. The intraoral tool cleaning system of claim 1, wherein the housing functions as a dovetail pin and the furrow functions as a dovetail slot, such that the housing engages with the furrow in a sliding dovetail joint.

4. The intraoral tool cleaning system of claim 1, wherein the backrest is at least as wide as the housing.

5. The intraoral tool cleaning system of claim 4, wherein the backrest is angled 45 degrees away from a plane defined by the vertical slot.

6. The intraoral tool cleaning system of claim 1, wherein the housing is made of a reusable material that is adapted to be sterilized; wherein the sponge is removable from the housing.

7. The intraoral tool cleaning system of claim 1, wherein the housing fitting of the base further includes a retention stop that limits the range in which the housing may engage with the furrow.

8. The intraoral tool cleaning system of claim 7, wherein the retention stop is a ridge in the furrow.

9. The intraoral tool cleaning system of claim 1, wherein the base is wider at its bottom than at its top.

10. The intraoral tool cleaning system of claim 9, wherein the base is shaped like a dome.

11. An intraoral tool cleaning system that enables a user to clean debris and fog from an intraoral mirror with a one-handed motion, comprising:
a disposable sponge adapted to remove particles from a mirror surface of the intraoral mirror by abrading the mirror surface, wherein the sponge is made of an absorbent foam material and is saturated with a surfactant fluid;
a disposable base that secures the sponge to a surface, wherein the surface is located on an appendage of the user; and
a fastener that adheres the sponge to the base, wherein the fastener includes an adhesive material applied to the sponge.

12. The intraoral tool cleaning system of claim 11, wherein the surface is located on the hand of the user.

13. The intraoral tool cleaning system of claim 12, wherein the surface is on a medical glove worn by the user.

14. The intraoral tool cleaning system of claim 13, wherein the base includes an adhesive patch that adheres to the glove.

15. The intraoral tool cleaning system of claim 12, wherein the surface is on a finger of the user; wherein the base is a ring that is worn on the finger.

16. The intraoral tool cleaning system of claim 11, wherein the surface is on a wrist of the user; wherein the base is a wrist strap that is worn on the wrist.

17. The intraoral tool cleaning system of claim 11 wherein the adhesive material of the fastener includes epoxy.

18. A method for cleaning debris and fog from a mirror surface of an intraoral mirror with a one-handed motion, comprising the steps of:
a. inserting the intraoral mirror into a vertical slot of a housing that contains a first disposable sponge, wherein the vertical slot is defined by the sponge and a back wall adjacent to the sponge; wherein the housing further includes an angled backrest extending from the back wall that guides insertion of the mirror surface into the vertical slot; wherein the sponge is saturated with a surfactant fluid and is adapted to remove particles from the mirror surface by abrading the mirror surface on the sponge;
b. abrading the mirror surface on the sponge with a one-handed motion such that the sponge removes particles from the mirror surface;
c. removing the intraoral mirror from the housing;
d. removing the sponge from the housing;
e. discarding the sponge; and
f. providing a second disposable sponge.

19. The method of claim 18, further comprising the step of discarding the housing; wherein the step of providing a second disposable sponge further includes providing a second housing that contains the second disposable sponge.

20. The method of claim 18, further comprising the step of sterilizing the housing; wherein the step of providing a second disposable sponge includes inserting the second disposable sponge in the sterilized housing.
